(19) United States
(12) Patent Application Publication
(10) Pub. No.: US 2026/0365809 A1
(43) Pub. Date: Nov. 19, 2026

(54) Joined Body Using Polyether Ether Ketone Resin, and Composite Material

(71) Applicant: Hitachi, Ltd., Chiyoda-ku, Tokyo (JP)

(72) Inventor: Tomio IWASAKI, Tokyo (JP)

(21) Appl. No.: 19/110,618

(22) PCT Filed: Sep. 28, 2023
(86) PCT No.: PCT/JP2023/035512

§ 371 Date: Mar. 11, 2025

(30) Foreign Application Priority Data
Nov. 4, 2022 (JP) 2022-177582

(51) Int. Cl. C08J 7/06; A61F 2/44; A61L 27/18; A61L 27/30; C08J 3/20; C08K 3/22

(52) U.S. Cl. CPC C08J 7/06; A61L 27/18; A61L 27/306; C08J 3/203; C08K 3/22; A61F 2/4455; A61F 2310/00389; A61L 2430/38; C08J 2371/10; C08K 2003/222; C08K 2003/2227; C08K 2003/2241

(57) ABSTRACT

The present invention provides a joined body of PEEK resin and a metal oxide and a composite material, having high adhesion strength between the PEEK resin and the metal oxide and hardly causing interfacial failure. The joined body is a joined body (1) in which a polyether ether ketone resin (2) and a metal oxide (3) are joined. A relative difference between a distance between benzene rings constituting a molecular chain of the polyether ether ketone resin (2) and a distance between metal atoms constituting a crystal structure of the metal oxide is 5% or less. An atomic density of the metal oxide on a crystal plane joined to the polyether ether ketone resin is 17 atoms/nm² or more. The composite material is a composite material which contains a metal oxide with a polyether ether ketone resin as a base material. A relative difference between a distance between benzene rings constituting a molecular chain of the polyether ether ketone resin and a distance between metal atoms constituting a crystal structure of the metal oxide is 5% or less.

### TECHNICAL FIELD

The present invention relates to a joined body of a polyether ether ketone (PEEK) resin and a metal oxide, and a composite material.

### BACKGROUND ART

Among thermoplastic resins, PEEK resin is a resin with high thermal stability and low reactivity, and has a property of being difficult to deteriorate. It also has excellent mechanical properties, chemical resistance, and water resistance, and is known as a material with high flame retardancy and biocompatibility. Since PEEK resin has these excellent properties, it is attracting attention as a material for medical use in contact with living tissue, a material for vehicles such as automobiles, and a material for aerospace applications.

In the medical field, PEEK resin is used as a material for implants, including intervertebral cages, artificial joints, and internal fixation devices for fractures. Generally, titanium dioxide or hydroxyapatite is coated on the surface of PEEK resin to improve the bonding properties with bone tissue. Patent Literature 1 describes a method of covering the surface of medical PEEK resin with titanium and forming a thin film of titanium dioxide on the surface of the titanium.

Furthermore, composite materials have been developed in which fillers are dispersed in a base material formed of PEEK resin. Conventionally, fiber-reinforced plastics (FRPs), in which carbon fibers or glass fibers are embedded in epoxy resin or phenolic resin, are known as lightweight composite materials with high strength and elastic modulus. By using thermoplastic PEEK resin instead of thermosetting resin, improvement in moldability and recyclability is expected.

Patent Literature 2 describes a porous PEEK resin suitable for implants and its manufacturing method. It is stated that bioactive ceramic can be mixed as particles in this PEEK resin. Examples of bioactive ceramics include tricalcium phosphate, hydroxyapatite, and biphasic calcium phosphate. In addition, as another technology, a technique for improving ultraviolet resistance by dispersing titanium dioxide in a base material is known.

### CITATION LIST

### Patent Literature

Patent Literature 1: US 2021/0079532 A1
Patent Literature 2: US 2012/0323339 A1

### SUMMARY OF INVENTION

### Technical Problem

Since PEEK resin has excellent heat resistance, mechanical properties, chemical resistance, and water resistance, it is used as a joined body in which other materials are joined to the surface, such as when titanium dioxide is coated, as a composite material in which other materials are dispersed in a base material, or the like. However, PEEK resin has the problem that it is difficult to join or combine with other materials.

Since PEEK resin has high thermal stability and low reactivity, joining using chemisorption and chemical modification to improve joining properties are difficult. When other materials are joined to the surface of PEEK resin or when other materials are dispersed in a base material formed of PEEK resin, if an external force or the like is applied, peeling due to interfacial failure easily occurs between the PEEK resin and the metal oxide.

Therefore, an object of the present invention is to provide a joined body of PEEK resin and a metal oxide and composite material having high adhesion strength between the PEEK resin and the metal oxide and hardly causing interfacial failure.

### Solution to Problem

In order to solve the above-described problems, a joined body according to the present invention is a joined body in which a polyether ether ketone resin and a metal oxide are joined, wherein a relative difference between a distance between benzene rings constituting a molecular chain of the polyether ether ketone resin and a distance between metal atoms constituting a crystal structure of the metal oxide is 5% or less; and an atomic density of the metal oxide on a crystal plane joined to the polyether ether ketone resin is 17 atoms/nm² or more.

Furthermore, a composite material according to the present invention is a composite material containing a metal oxide with a polyether ether ketone resin as a base material, wherein a relative difference between a distance between benzene rings constituting a molecular chain of the polyether ether ketone resin and a distance between metal atoms constituting a crystal structure of the metal oxide is 5% or less.

### Advantageous Effects of Invention

According to the present invention, it is possible to provide a joined body of PEEK resin and a metal oxide and composite material having high adhesion strength between the PEEK resin and the metal oxide and hardly causing interfacial failure.

### BRIEF DESCRIPTION OF DRAWINGS

FIG. 1 is a cross-sectional view schematically showing an example of a joined body according to an embodiment of the present invention.

FIG. 2 is a cross-sectional view schematically showing a usage example of the joined body according to the embodiment of the present invention.

FIG. 3 is a cross-sectional view schematically showing an example of a joined body according to an embodiment of the present invention.

FIG. 4 is a cross-sectional view schematically showing an example of a composite material according to an embodiment of the present invention.

FIG. 5 is a perspective view schematically showing a metal oxide dispersed in the composite material according to the embodiment of the present invention.

FIG. 6 is a diagram showing analysis results of the relationship between the ratio of the (110) plane to the total surface area of titanium dioxide and the yield strength of the composite material.

FIG. 7 is a diagram showing analysis results of the relationship between the ratio of the (001) plane to the total surface area of aluminum oxide and the yield strength of the composite material.

FIG. 8 is a diagram showing analysis results of the relationship between the ratio of the (111) plane to the total surface area of magnesium oxide and the yield strength of the composite material.

FIG. 9 is a cross-sectional view schematically showing an example of a composite material according to an embodiment of the present invention.

FIG. 10 is a diagram showing analysis results of the atomic arrangement of PEEK resin not using a metal oxide.

FIG. 11 is a diagram showing analysis results of the atomic arrangement of PEEK resin using titanium dioxide.

FIG. 12 is a diagram showing analysis results of the atomic arrangement of PEEK resin using aluminum oxide.

FIG. 13 is a diagram showing analysis results of the atomic arrangement of PEEK resin using magnesium oxide.

### DESCRIPTION OF EMBODIMENTS

Hereinafter, a joined body of a polyether ether ketone (PEEK) resin and a metal oxide, and a composite material, as well as manufacturing methods thereof according to an embodiment of the present invention will be described with reference to the drawings. In the following drawings, the same components are denoted by the same reference numerals, and redundant description will be omitted.

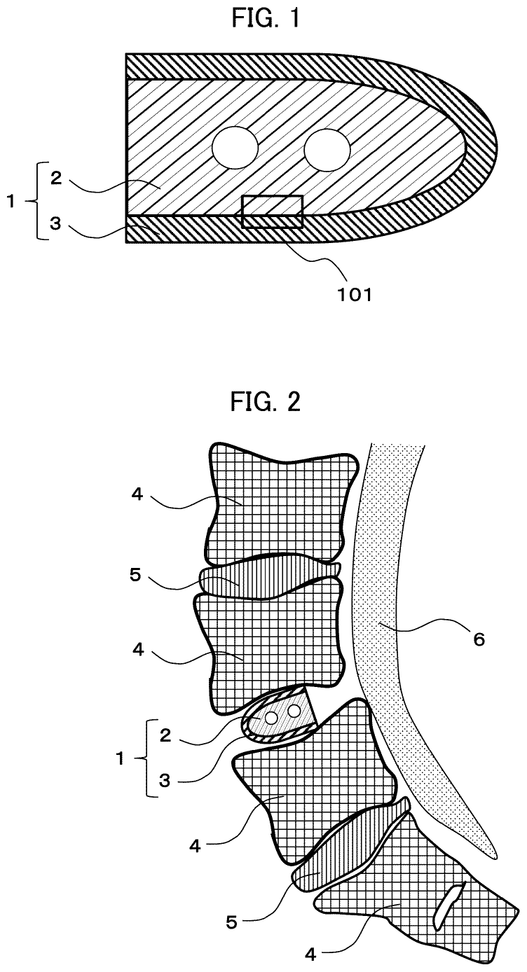

FIG. 1 is a cross-sectional view schematically showing an example of a joined body according to the embodiment of the present invention. FIG. 2 is a cross-sectional view schematically showing a usage example of the joined body according to the embodiment of the present invention. FIGS. 1 and 2 show an intervertebral cage as an example of a joined body in which PEEK resin and a metal oxide are joined. FIG. 2 shows a cross-sectional view around the human spine where the intervertebral cage is used.

In FIGS. 1 and 2, reference numeral 1 denotes a joined body forming an intervertebral cage, reference numeral 2 denotes PEEK resin, reference numeral 3 denotes a metal oxide, reference numeral 4 denotes a vertebral body, reference numeral 5 denotes an intervertebral disc, reference numeral 6 denotes bone marrow, and reference numeral 101 denotes an interface between the PEEK resin and the metal oxide.

As shown in FIG. 1, the joined body 1 according to the present embodiment has a structure in which PEEK resin 2 and metal oxide 3 are joined to each other. The PEEK resin 2 and the metal oxide 3 are directly joined to each other without interposing another substance by adhesion mainly based on physical interaction occurring between atoms. As the metal oxide 3, a predetermined metal oxide having a predetermined distance between metal atoms 5 and exhibiting an action of aligning the atomic arrangement of the PEEK resin is joined.

The joined body 1 shown in FIG. 1 forms an intervertebral cage. The intervertebral cage is a type of implant that is embedded in the body and is used for the treatment of spinal canal stenosis, scoliosis, and the like. As shown in FIG. 2, there is an intervertebral disc 5 between the vertebral bodies 4 constituting the spine. The intervertebral cage is inserted between the vertebral bodies 4 as a substitute for the intervertebral disc 5 when the intervertebral disc 5 is removed due to damage or the like.

The intervertebral cage functions as a spacer that fixes the height between the vertebral bodies 4 and a cushion that stabilizes the spine by buffering between the vertebral bodies 4. In FIGS. 1 and 2, the intervertebral cage is provided in a cage-like structure. The inside of the intervertebral cage is filled with a bone graft or the like depending on the treatment method. The joined body 1 in which the PEEK resin 2 and the metal oxide 3 are joined to each other can be used as a material for such an intervertebral cage.

The intervertebral cage can be provided in an appropriate shape and structure. In FIGS. 1 and 2, through-holes are formed on the side surface of the intervertebral cage. The intervertebral cage can also be provided in a form in which through-holes are not formed on the side surface, a configuration in which lattices are provided in upper and lower openings or through-holes on the side surface, or a configuration in which irregularities are formed on the upper and lower surfaces around the openings.

In the joined body 1 according to the present embodiment, the metal oxide 3 used is one having a predetermined distance between the metal atoms and a relative difference of 5% or less between a distance between the benzene rings constituting the molecular chain of the PEEK resin 2 and a distance between the metal atoms constituting the crystal structure of the metal oxide 3. The relative difference is the absolute value of the difference in distance and means the ratio of the difference between the relatively longer interval length and the relatively shorter interval length to the relatively shorter interval length ((longer interval length-shorter interval length)/shorter interval length) [%].

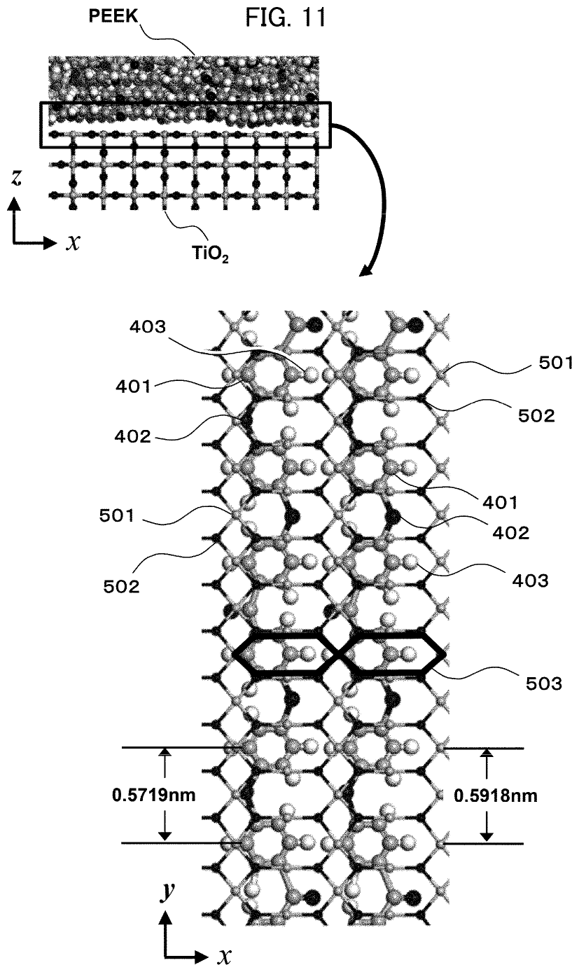

In this specification, the distance between the benzene rings constituting the molecular chain of the PEEK resin means the distance between the centers of the benzene rings connected with an intervening ether group. The distance between the benzene rings is determined within one polyether ether ketone unit composed of phenylene, ether, and ketone. The distance between the benzene rings is determined in a conformation in which the ether group has a predetermined bond angle and the benzene rings are oriented in parallel on the same plane (see FIGS. 11 to 13).

In this specification, the distance between the metal atoms constituting the crystal structure of the metal oxide means the distance between periodically arranged metal atoms forming the crystal of the metal oxide. The distance between metal atoms can be determined between any type of metal atoms, between metal atoms located at any metal site, between metal atoms arranged in any crystal orientation, or between metal atoms in an arbitrary proximity state, as the distance corresponding to the distance between benzene rings of PEEK resin.

The metal atom in an arbitrary proximity state means a metal atom located by sandwiching an arbitrary number of metal atoms with respect to a metal atom serving as a starting point of distance, among multiple metal atoms periodically arranged on the same crystal plane in the crystal lattice of the metal oxide. For example, the distance from the metal atom serving as the starting point of the distance to the first nearest metal atom closest to the metal atom, the distance to the second nearest metal atom located by sandwiching one metal atom with respect to the metal atom, and the like can be compared with the distance between the benzene rings of the PEEK resin.

Conventionally, intervertebral cages have been developed which are formed of PEEK resin and have titanium dioxide or hydroxyapatite (HAp) coated on the surface thereof. It is known that when an implant is formed only of PEEK resin, surrounding bone tissue is difficult to achieve osseointegration. Since titanium dioxide and HAp have good bonding properties with bone tissue and have similar properties to bone tissue, titanium dioxide or HAp is coated on the surface of PEEK resin.

However, PEEK resin is known to have low joining properties with other materials. When directly joining PEEK resin and a metal oxide, there is a problem that high joining strength cannot be obtained because the adhesion phenomenon hardly works. Since PEEK resin has high thermal stability and low reactivity, chemical adsorption and chemical modification are also difficult. Even if a metal oxide is simply coated, peeling due to interfacial failure easily occurs when an external force or the like is applied.

On the other hand, when the relative difference between the distance between the benzene rings of the PEEK resin 2 and the distance between the metal atoms of the metal oxide 3 is 5% or less, at the interface 101 between the PEEK resin 2 and the metal oxide 3, the carbon atoms of the benzene rings and the metal atoms of the metal oxide 3 can be arranged in close proximity to each other to form a strong interaction. The molecular chains of the PEEK resin 2 are in a state where they can be aligned with an appropriate orientation and conformation with respect to the crystal structure of the metal oxide 3.

As a result, a lattice-matched interface is formed between the PEEK resin 2 and the metal oxide 3 by the periodic arrangement of the carbon atoms of the benzene rings and the metal atoms of the metal oxide 3 close to each other. Since lattice mismatch is reduced between the crystal phase formed by the molecular chains of the PEEK resin 2 and the crystal structure of the metal oxide 3, a highly adhesive state that is stable in terms of electronic state and energy state is obtained. Therefore, the PEEK resin 2 and the metal oxide 3 can be joined with high joining strength.

Further, another molecular chain of the PEEK resin 2 can be aligned with an appropriate orientation and conformation with respect to the molecular chain of the PEEK resin 2 aligned on the surface of the metal oxide 3. The action of the metal oxide 3 to align the atomic arrangement of the PEEK resin extends not only to the interface 101 between the PEEK resin 2 and the metal oxide 3 but also to a region away from the interface 101. Therefore, the adhesion strength between the molecular chains of the PEEK resin 2 can be increased even in a region away from the interface 101.

A resin having an appropriate molecular weight or degree of polymerization can be used as the PEEK resin 2. As the PEEK resin 2, a homopolymer containing only polyether ether ketone units may be used, or a copolymer obtained by polymerizing polyether ketone units, polyether ether ketone ketone units, or other monomers may be used. However, as the PEEK resin 2, it is preferable to use a homopolymer from the viewpoint of reducing lattice mismatch.

The PEEK resin 2 may or may not contain additives. Examples of the additive include fillers, plasticizers, flame retardants, and the like. As the additive, a predetermined metal oxide exhibiting an action of aligning the atomic arrangement of the PEEK resin can also be added as in the composite material described later.

Examples of the filler include silica, talc, mica, clay, calcium carbonate, potassium titanate, carbon, glass fiber, carbon fiber, organic fiber, and the like. Examples of the plasticizer include phthalate esters, adipate esters, trimellitic acid esters, phosphate esters, and the like. Examples of the flame retardant include aluminum hydroxide, magnesium hydroxide, magnesium carbonate, ammonium polyphosphate, ammonium carbonate, antimony oxide, boric acid compounds, molybdenum compounds, bromine compounds, red phosphorus, phosphate esters, melamine compounds, triazine compounds, guanidine compounds, and the like.

As the metal oxide 3, as long as the relative difference between the distance between the benzene rings of the PEEK resin 2 and the distance between the metal atoms of the metal oxide 3 is 5% or less, an oxide of an appropriate metal can be joined. The metal oxide 3 may be an oxide containing one kind of metal as a main component or a composite oxide containing multiple kinds of metals as main components.

As the metal oxide 3, a single crystal may be joined or a polycrystal may be joined as long as a lattice-matched interface is formed with the PEEK resin 2. However, from the viewpoint of joining the PEEK resin 2 and the metal oxide 3 with high joining strength, it is preferable that the area ratio of the crystal plane in which the relative difference between the distance between the benzene rings of the PEEK resin 2 and the distance between the metal atoms of the metal oxide 3 is 5% or less is large, and it is preferable to join a single crystal as the metal oxide 3.

The thickness of the metal oxide 3 in the direction perpendicular to the surface joined to the PEEK resin 2 is preferably 2 nm or more and 100 μm or less. When the thickness is 2 nm or more, since a lattice-matched interface is formed by many atoms between the PEEK resin 2 and the metal oxide 3, the joining strength between the PEEK resin 2 and the metal oxide 3 can be further improved. Further, when the thickness is 100 μm or less, peeling of the metal oxide 3 due to stress concentration or cohesive failure can be reduced.

The atomic density on the crystal plane of the metal oxide 3 joined to the PEEK resin 2 is preferably 17 atoms/nm² or more. Even if the distance between the metal atoms of the metal oxide 3 is a predetermined length, if the density of the metal atoms is low, interaction with the PEEK resin 2 becomes difficult to form. On the other hand, when the atomic density is 17 atoms/nm² or more, since a lattice-matched interface is formed between the PEEK resin 2 and the metal oxide 3 by high-density atoms, the joining strength between the PEEK resin 2 and the metal oxide 3 can be further improved.

In the joined body 1 according to the present embodiment, preferred examples of the metal oxide 3 joined to the PEEK resin 2 are as follows (1) to (3).
(1) The metal oxide 3 is titanium dioxide (titania: TiO₂). The crystal plane of the metal oxide 3 joined to the PEEK resin 2 is a crystal plane having a Miller index of (110) of titanium dioxide having a rutile structure, or a crystal plane equivalent thereto. The atomic density on the crystal plane of the titanium dioxide having a rutile structure joined to the PEEK resin 2 is preferably 17 atoms/nm² or more.
(2) The metal oxide 3 is aluminum oxide (alumina: Al₂O₃). The crystal plane of the metal oxide 3 joined to the PEEK resin 2 is a crystal plane having a Miller index of (001) of aluminum oxide, or a crystal plane equivalent thereto. The atomic density on the crystal plane of the aluminum oxide joined to the PEEK resin 2 is preferably 17 atoms/nm² or more.
(3) The metal oxide 3 is magnesium oxide (magnesia: MgO). The crystal plane of the metal oxide 3 joined to the PEEK resin 2 is a crystal plane having a Miller index of (111) of magnesium oxide, or a crystal plane equivalent thereto. The atomic density on the crystal plane of the magnesium oxide joined to the PEEK resin 2 is preferably 17 atoms/nm² or more.

According to the metal oxides 3 of (1) to (3), the relative difference between the distance between the benzene rings constituting the molecular chain of the PEEK resin and the distance between the metal atoms constituting the crystal structure of the metal oxide is 5% or less. Therefore, by the action of aligning the atomic arrangement of the PEEK resin, a lattice-matched interface can be formed between the PEEK resin and the metal oxide, and the joining strength between them can be increased. Further, the adhesion strength between the molecular chains of the PEEK resin can be increased in a region away from the interface.

Tables 1 to 3 show the results of measuring the joining strength between the PEEK resin and the metal oxide by a peel test. In the peel test, the joining strength of the metal oxide to PEEK resin or hydroxyapatite (HAp) was actually measured. The joining strength of each main crystal plane was measured for titanium dioxide having a rutile structure, aluminum oxide, and magnesium oxide.

A test piece of a joined body in which PEEK resin and a metal oxide were joined was used for the peel test. The test piece of the joined body was produced by directly bonding PEEK resin to a crystal plane having a predetermined Miller index on the surface of a single crystal of the metal oxide. The test piece was in a strip shape with a width of 25 mm.

In the peel test, after an adhesive tape was bonded to the surface of the PEEK resin, one end of the adhesive tape was peeled off at a 90-degree angle by a tensile tester to measure the peel force. Then, based on the average value of the measured peel force, the joining strength [kN/m] per unit width of the test piece was determined.

TABLE 1

Titanium Dioxide (TiO₂)

| | (100) | (001) | (110) | (111) |
|---|---|---|---|---|
| Joining Strength with | 2.96 | 3.71 | 9.48 | 3.95 |
| PEEK Resin (Peel | | | | |
| Strength) [kN/m] | | | | |
| Joining Strength with | 6.39 | 8.63 | 12.7 | 9.42 |
| HAp (Peel Strength) | | | | |
| [kN/m] | | | | |

Table 1 shows the results obtained using titanium dioxide having a rutile structure as the metal oxide. In the case of titanium dioxide having a rutile structure, the joining strength of the (110) plane was significantly higher for both PEEK resin and HAp than that of the (100) plane, (001) plane, and (210) plane.

TABLE 2

Aluminum Oxide (Al₂O₃)

| | (100) | (001) | (110) | (210) |
|---|---|---|---|---|
| Joining Strength with | 3.17 | 8.94 | 3.17 | 1.94 |
| PEEK Resin (Peel | | | | |
| Strength) [kN/m] | | | | |
| Joining Strength with | 6.74 | 11.3 | 6.74 | 5.39 |
| HAp (Peel Strength) | | | | |
| [kN/m] | | | | |

Table 2 shows the results obtained using aluminum oxide as the metal oxide. In the case of aluminum oxide, the joining strength of the (001) plane was significantly higher for both PEEK resin and HAp than that of the (100) plane, (110) plane, and (210) plane.

TABLE 3

Magnesium Oxide (MgO)

| | (100) | (001) | (110) | (111) |
|---|---|---|---|---|
| Joining Strength with | 4.58 | 4.58 | 4.52 | 8.36 |
| PEEK Resin (Peel | | | | |
| Strength) [kN/m] | | | | |
| Joining Strength with | 6.29 | 6.29 | 6.23 | 9.86 |
| HAp (Peel Strength) | | | | |
| [kN/m] | | | | |

Table 3 shows the results obtained using magnesium oxide as the metal oxide. In the case of magnesium oxide, the joining strength of the (111) plane was significantly higher for both PEEK resin and HAp than that of the (100) plane, (001) plane, and (110) plane.

As shown in Tables 1 to 3, the joining strength between the metal oxide and the PEEK resin and the joining strength between the metal oxide and HAp showed similar tendencies with respect to the type of crystal plane represented by the same Miller index. This is because the distance between calcium atoms of HAp is close to the distance between benzene rings of PEEK resin. It can be said that under the condition where the lattice mismatch between the metal oxide and the PEEK resin is reduced, the lattice mismatch between the metal oxide and HAp is also reduced.

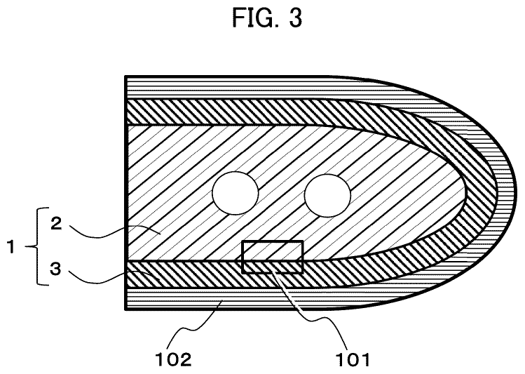

FIG. 3 is a cross-sectional view schematically showing an example of a joined body according to the embodiment of the present invention. FIG. 3 shows an intervertebral cage as an example of a joined body in which PEEK resin and a metal oxide are joined.

As shown in FIG. 3, an HAp film 102 can also be formed on the surface of the joined body 1 in which the PEEK resin 2 and the metal oxide 3 are joined to each other.

The HAp film 102 is mainly formed of hydroxyapatite (HAp) (Ca₁₀(PO₄)₆(OH)₂). The HAp film 102 may have calcium deficiency or a different phase such as calcium phosphate. The HAp film 102 can be formed on the surface of the metal oxide 3 opposite to the surface joined to the PEEK resin 2. By covering the surface of the metal oxide 3 with the HAp film 102, the bonding properties between the joined body 1 and bone tissue can be improved.

The thickness of the HAp film 102 is preferably 2 nm or more and 50 μm or less. When the thickness is 2 nm or more, a lattice-matched interface is formed by many atoms between the metal oxide 3 and the HAp film 102, so that the joining strength between the metal oxide 3 and the HAp film 102 can be further improved. Further, when the thickness is 50 μm or less, it is possible to reduce peeling of the HAp film 102 due to stress concentration or cohesive failure.

As shown in Tables 1 to 3, the joining strength between the metal oxide and HAp is significantly increased when the metal oxide has a predetermined crystal plane. Therefore, from the viewpoint of joining the metal oxide 3 and the HAp film 102 with higher joining strength, the HAp film 102 is preferably formed on the (110) plane of titanium dioxide having a rutile structure, the (001) plane of aluminum oxide, or the (111) plane of magnesium oxide.

Next, a method for manufacturing the joined body according to the present embodiment in which the PEEK resin and the metal oxide are joined to each other will be described.

The joined body according to the present embodiment can be manufactured by a manufacturing method of film-forming a predetermined metal oxide exhibiting an action of aligning the atomic arrangement of the PEEK resin on the surface of the PEEK resin. The metal oxide is formed as a thin film having a predetermined crystal structure so that the relative difference between the distance between the benzene rings of the PEEK resin and the distance between the metal atoms of the metal oxide is 5% or less.

The manufacturing method of the joined body according to the present embodiment includes a film forming step of film-forming the metal oxide on the surface of the PEEK resin, and a heat treatment step of heat-treating the PEEK resin on which the metal oxide is film-formed to improve the crystal orientation of the metal oxide. When covering the surface of the metal oxide with the HAp film, after the heat treatment step, a film forming step of forming the HAp film on the surface of the metal oxide is performed.

As a film forming method for film-forming a metal oxide on the surface of the PEEK resin, a transfer film forming method of transferring a metal oxide film-formed on a substrate to the surface of the PEEK resin, or a direct film forming method of directly film-forming the metal oxide on the surface of the PEEK resin can be used.

When the transfer film forming method is used, first, a substrate of a single crystal formed of a metal oxide having a predetermined distance between the metal atoms and exhibiting an action of aligning the atomic arrangement of the PEEK resin is prepared. For example, a substrate having a surface of the (110) plane of titanium dioxide having a rutile structure, the (001) plane of aluminum oxide, the (111) plane of magnesium oxide, or the like is prepared.

Subsequently, an HAp film formed of HAp is formed on the surface of the single crystal substrate formed of the metal oxide. As a film forming method, a physical vapor deposition method such as pulsed laser deposition (PLD) method or sputtering method, a liquid phase synthesis method, a plasma spraying method, or the like can be used. Since the distance between calcium atoms of HAp is close to the distance between metal atoms of the metal oxide, a lattice-matched HAp film is formed.

Subsequently, a metal oxide having a predetermined distance between the metal atoms and exhibiting an action of aligning the atomic arrangement of the PEEK resin is film-formed on the surface of the HAp film formed on the substrate. As the film forming method, a physical vapor deposition method such as an arc ion plating (AIP) method or a sputtering method can be used. Since the distance between calcium atoms of HAp is close to the distance between benzene rings of PEEK resin, a lattice-matched metal oxide is film-formed.

Next, the metal oxide film-formed on the substrate is peeled from the substrate. The metal oxide film-formed on the substrate may be peeled at the interface with the HAp film, or may be peeled together with the HAp film at the interface with the single crystal substrate. For example, a method can be used in which, after a transfer plate to which an adhesive tape is attached is bonded to the surface of the metal oxide film-formed on the substrate, the metal oxide is peeled from the single crystal substrate at the interface with the HAp film or at the interface with the single crystal substrate.

Subsequently, the metal oxide peeled from the substrate is transferred to and covers the surface of the PEEK resin molded into a predetermined shape. As a method of transferring the thin film of the metal oxide, for example, a method can be used in which, after the metal oxide bonded to the transfer plate via the adhesive tape is bonded to the surface of the PEEK resin, the transfer plate is peeled from the metal oxide. The transfer plate bonded via the adhesive tape can be peeled off with a solvent. The surface of the PEEK resin can be subjected to surface treatment such as plasma treatment or corona discharge treatment in advance.

Subsequently, the PEEK resin on which the metal oxide is film-formed is heat-treated to improve the crystal orientation of the metal oxide. The heat treatment temperature is preferably 100 °C or more and 400 °C or less. When the heat treatment temperature is 100 °C or more, it is possible to reduce disorder of the crystal structure of the metal oxide and to increase the area ratio of the predetermined crystal structure in which the distance between metal atoms is a predetermined length. When the heat treatment temperature is 400 °C or less, thermal decomposition of the PEEK resin can be avoided.

When the crystal orientation of the metal oxide is improved by heat treatment, the lattice mismatch at the interface is reduced, and a lattice-matched interface is formed between the PEEK resin and the metal oxide. By such heat treatment, the joined body 1 in which the PEEK resin 2 and the metal oxide 3 are joined to each other can be obtained. Further, when the metal oxide is peeled from the substrate together with the HAp film and covered on the surface of the PEEK resin, a joined body in which the surface of the metal oxide is covered with the HAp film can be obtained.

According to such a transfer film forming method, since the metal oxide film-formed on the substrate is transferred to the surface of the PEEK resin, a predetermined metal oxide having a predetermined distance between the metal atoms and exhibiting an action of aligning the atomic arrangement of the PEEK resin can be film-formed while suppressing the thermal load on the PEEK resin. Since the final heat treatment only needs to reduce the disorder of the crystal structure of the metal oxide, the heat treatment temperature can be reduced to a low temperature.

Further, according to such a film forming method, in the step of peeling the metal oxide film-formed on the substrate, only the metal oxide can be peeled at the interface with the HAp film to manufacture the joined body 1 in which the PEEK resin 2 and the metal oxide 3 are joined to each other, or the metal oxide can be peeled together with the HAp film at the interface with the single crystal substrate to manufacture the joined body 1 in which the surface of the metal oxide 3 is covered with the HAp film 102. When the metal oxide is peeled together with the HAp film, it becomes possible to manufacture the joined body 1, covered with the HAp film 102 that easily achieves osseointegration with bone tissue, by omitting the film forming step.

On the other hand, when the direct film forming method is used, first, a metal oxide having a predetermined distance between the metal atoms and exhibiting an action of aligning the atomic arrangement of the PEEK resin is film-formed on the surface of the PEEK resin molded into a predetermined shape. The surface of the PEEK resin can be subjected to surface treatment such as plasma treatment or corona discharge treatment in advance. As the film forming method, a physical vapor deposition method such as an arc ion plating (AIP) method or a sputtering method can be used.

Subsequently, the PEEK resin on which the metal oxide is film-formed is heat-treated to improve the crystal orientation of the metal oxide. The heat treatment temperature is preferably 100 °C or more and 400 °C or less, as in the case of using the transfer film forming method.

When the crystal orientation of the metal oxide is improved by heat treatment, the lattice mismatch at the interface is reduced, and a lattice-matched interface is formed between the PEEK resin and the metal oxide. By such heat treatment, the joined body 1 in which the PEEK resin 2 and the metal oxide 3 are joined to each other can be obtained.

Subsequently, an HAp film formed of HAp is formed on the surface of the metal oxide film-formed on the surface of the PEEK resin. As a film forming method, a physical vapor deposition method such as pulsed laser deposition (PLD) method or sputtering method, a liquid phase synthesis method, a plasma spraying method, or the like can be used. Note that when the HAp film is not necessary, the formation of the HAp film can be omitted.

According to such a direct film forming method, since the metal oxide is directly film-formed on the surface of the PEEK resin, a predetermined metal oxide having a predetermined distance between the metal atoms and exhibiting an action of aligning the atomic arrangement of the PEEK resin can be formed with less steps. Further, even when the PEEK resin is molded into a complicated shape, the metal oxide exhibiting a predetermined crystal orientation can be film-formed over a wide range.

At the time of forming the metal oxide or the HAp film, as described in the literature (Masazumi Okido, et al., Evaluation of the Hydroxyapatite Film Coating on Titanium Cathode by QCM, Materials Transactions, Vol. 43, No. 12 (2002) p. 3010-3014), it is preferable to monitor the film thickness by a quartz crystal microbalance (QCM) method. The thickness of the metal oxide is preferably 2 nm or more and 100 μm or less. The thickness of the HAp film is preferably 2 nm or more and 50 μm or less.

When the AIP method is used as the film forming method for film-forming the metal oxide, film formation is performed by arc discharge in a vacuum atmosphere using PEEK resin as a film formation target, a metal constituting the metal oxide as an evaporation source, and oxygen gas or a mixed gas of oxygen and an inert gas as a reaction gas. The target current applied to the evaporation source is preferably 100 A or more. The substrate bias voltage applied to the film formation target is preferably 60 V or more.

For example, when improving the crystal orientation of titanium dioxide having a rutile structure and increasing the area ratio of the (110) plane, a high temperature of about 800 °C or more is required. When the temperature at the time of film formation is low, the ratio of the anatase structure or the amorphous structure tends to increase, and the area ratio of the (110) plane tends to decrease. If the target current is 100 A or more and the bias voltage is 60 V or more, the temperature at the time of film formation becomes high, so that the crystal orientation of the metal oxide can be appropriately increased.

According to the joined body according to the present embodiment described above, the relative difference between the distance between the benzene rings of the PEEK resin and the distance between the metal atoms of the metal oxide is 5% or less, and the metal oxide exhibiting an action of aligning the atomic arrangement of the PEEK resin is joined to the PEEK resin, so that a lattice-matched interface can be formed between the PEEK resin and the metal oxide. Therefore, it is possible to obtain a joined body having high adhesion strength between the PEEK resin and the metal oxide, and hardly causing interfacial failure at the interface between the PEEK resin and the metal oxide. Further, since the action of aligning the atomic arrangement of the PEEK resin extends to a region away from the interface, the yield strength of the base material formed of the PEEK resin can be improved.

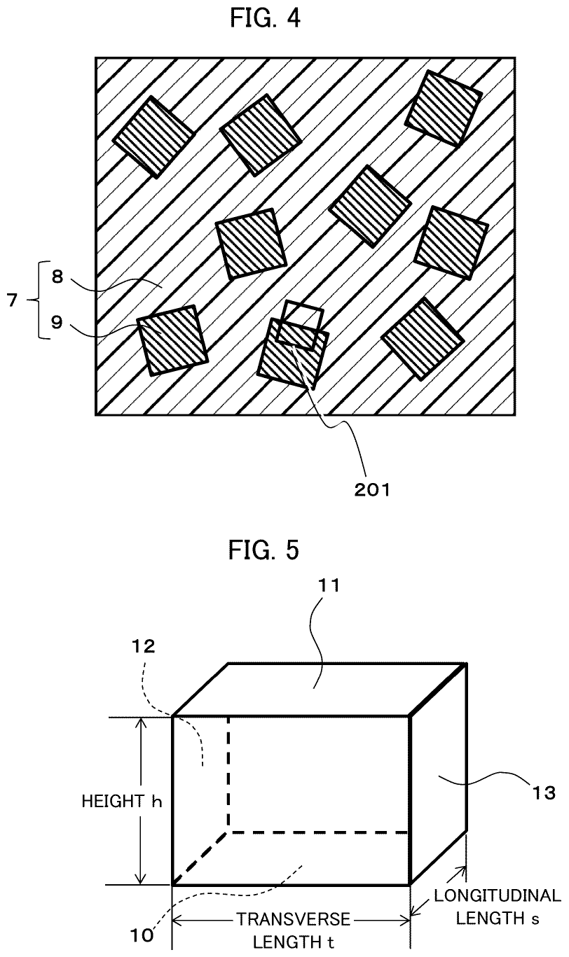

FIG. 4 is a cross-sectional view schematically showing an example of a composite material according to the embodiment of the present invention. FIG. 4 shows an example of a composite material containing a metal oxide with PEEK resin as a base material. In FIG. 4, reference numeral 7 denotes the composite material, reference numeral 8 denotes PEEK resin, reference numeral 9 denotes a metal oxide, and reference numeral 201 denotes an interface between the PEEK resin and the metal oxide.

As shown in FIG. 4, the composite material 7 according to the present embodiment includes at least PEEK resin 8 forming a base material and metal oxide 9 dispersed in the base material. As the metal oxide 9, a predetermined metal oxide having a predetermined distance between metal atoms and exhibiting an action of aligning the atomic arrangement of the PEEK resin is blended.

The composite material 7 according to the present embodiment can be molded into an appropriate shape depending on the application and the like. In the composite material 7 according to the present embodiment, the metal oxide 9 used is one having a predetermined distance between the metal atoms and a relative difference of 5% or less between the distance between the benzene rings constituting the molecular chain of the PEEK resin 8 and the distance between the metal atoms constituting the crystal structure of the metal oxide 9.

When such a metal oxide 9 is dispersed, a lattice-matched interface is formed between the PEEK resin 8 and the metal oxide 9 by periodically arranging the carbon atoms of the benzene rings and the metal atoms of the metal oxide 9 in close proximity to each other. Since the lattice mismatch is reduced between the crystal phase formed by the molecular chains of the PEEK resin 8 and the crystal structure of the metal oxide 9, a close contact state with high adhesion strength, which is stable electronically and energetically, is obtained.

As a result, at the interface 201 between the PEEK resin 8 and the metal oxide 9, the molecular chains of the PEEK resin 8 can be aligned with an appropriate orientation and conformation with respect to the crystal structure of the metal oxide 9. Another molecular chain of the PEEK resin 8 can be aligned with an appropriate orientation and conformation with respect to the molecular chain of the PEEK resin 8 aligned on the surface of the metal oxide 9.

Therefore, the action of the metal oxide 9 to align the atomic arrangement of the PEEK resin reaches not only the interface 201 between the PEEK resin 8 and the metal oxide 9, but also the region away from the interface 201. The action of the PEEK resin 8 to align the atomic arrangement is obtained over a wide range starting from the metal oxide 9. Generally, it is difficult to obtain sufficiently high rigidity with only PEEK resin, but since the molecular chains of the PEEK resin 8 can be aligned and the adhesion strength between the molecular chains of the PEEK resin 8 can be increased, the yield strength of the composite material 7 can be improved.

A resin having an appropriate molecular weight or degree of polymerization can be used as the PEEK resin 8. As the PEEK resin 8, a homopolymer containing only polyether ether ketone units may be used, or a copolymer obtained by polymerizing polyether ketone units, polyether ether ketone ketone units, or other monomers may be used. However, as the PEEK resin 8, it is preferable to use a homopolymer from the viewpoint of reducing lattice mismatch.

The PEEK resin 8 may or may not contain additives other than the metal oxide 9 exhibiting an action of aligning the atomic arrangement of the PEEK resin. Examples of the additives other than the metal oxide 9 include fillers, plasticizers, flame retardants, and the like. As the filler, plasticizer, and flame retardant, the same types as those of the joined body 1 can be used.

As the metal oxide 9, as long as the relative difference between the distance between the benzene rings of the PEEK resin 8 and the distance between the metal atoms of the metal oxide 9 is 5% or less, an oxide of an appropriate metal can be blended. The metal oxide 9 may be an oxide containing one kind of metal as a main component or a composite oxide containing multiple kinds of metals as main components.

As the metal oxide 9, one exhibiting an action of preventing ultraviolet rays in addition to the action of aligning the atomic arrangement of the PEEK resin is preferable. When the composite material 7 is used for aerospace applications or outdoors, the PEEK resin 8 may deteriorate due to the influence of ultraviolet rays. Therefore, the metal oxide 9 is preferably of a type exhibiting an action of reflecting, scattering, or absorbing ultraviolet rays, and titanium dioxide in the rutile phase is preferable as compared with aluminum oxide and magnesium oxide.

As the metal oxide 9, a single crystal or a polycrystal may be blended as long as lattice mismatch with the PEEK resin 8 is prevented. However, from the viewpoint of improving the mechanical properties of the composite material 7 over a wide range, it is preferable that the area ratio of the crystal plane in which the relative difference between the distance between the benzene rings of the PEEK resin 8 and the distance between the metal atoms of the metal oxide 9 is 5% or less is large, and it is preferable to blend a single crystal as the metal oxide 9.

The concentration of the metal oxide 9 is preferably 0.2 at % or more and 38 at % or less. When the concentration is 0.2 at % or more, the starting points of the action of aligning the atomic arrangement of the PEEK resin increase. Further, when the concentration is 38 at % or less, embrittlement of the base material formed of the PEEK resin is avoided, and the starting points of the action of aligning the atomic arrangement of the PEEK resin can be discretely dispersed. When the concentration is 0.2 at % or more and 38 at % or less, the actions from a large number of starting points reach a region away from the interface 201 between the PEEK resin 8 and the metal oxide 9 independently without interfering with each other, so that the molecular chains of the PEEK resin are aligned over a wide range of the base material, and a high effect of improving the yield strength can be obtained.

The average particle size of the metal oxide 9 is preferably 2 nm or more and 10 μm or less. When the average particle size is 2 nm or more, the crystallinity becomes good and variation in particle size is suppressed, so that a crystal plane having a predetermined distance between metal atoms is easily obtained. Further, when the average particle size is 10 μm or less, embrittlement of the base material formed of the PEEK resin is avoided, and the specific surface area of the metal oxide 9 increases. When the average particle size is 2 nm or more and 10 μm or less, the action of aligning the atomic arrangement of the PEEK resin can reach a wide range away from the interface 201 between the PEEK resin 8 and the metal oxide 9, and the yield strength of the base material can be improved.

The atomic density on the crystal plane of the metal oxide 9 in contact with the PEEK resin 8 is preferably 17 atoms/nm² or more. Even if the distance between the metal atoms of the metal oxide 9 is a predetermined length, if the density of the metal atoms is low, interaction with the PEEK resin 8 is difficult to form. On the other hand, when the atomic density is 17 atoms/nm² or more, a lattice-matched interface is formed between the PEEK resin 8 and the metal oxide 9 by high-density atoms, so that the action of aligning the atomic arrangement of the PEEK resin can reach a wide range away from the interface 201 between the PEEK resin 8 and the metal oxide 9, and the yield strength of the base material can be improved. In the composite material 7 according to the present embodiment, preferred examples of the metal oxide 9 dispersed in the base material formed of the PEEK resin 8 are as follows (4) to (6).
(4) The metal oxide 9 is titanium dioxide (titania: TiO₂). The surface of the metal oxide 9 in contact with the PEEK resin 8 includes a crystal plane having a Miller index of (110) of titanium dioxide having a rutile structure, or a crystal plane equivalent thereto. The metal oxide 9 is preferably one in which the area ratio of the (110) plane is the largest among all crystal planes detected in the metal oxide 9. The atomic density on the crystal plane of the titanium dioxide having a rutile structure in contact with the PEEK resin 8 is preferably 17 atoms/nm² or more.
(5) The metal oxide 9 is aluminum oxide (alumina: Al₂O₃). The surface of the metal oxide 9 in contact with the PEEK resin 8 includes a crystal plane having a Miller index of (001) of aluminum oxide, or a crystal plane equivalent thereto. The metal oxide 9 is preferably one in which the area ratio of the (001) plane is the largest among all crystal planes detected in the metal oxide 9. The atomic density on the crystal plane of the aluminum oxide in contact with the PEEK resin 8 is preferably 17 atoms/nm² or more.
(6) The metal oxide 9 is magnesium oxide (magnesia: MgO). The surface of the metal oxide 9 in contact with the PEEK resin 8 includes a crystal plane having a Miller index of (111) of magnesium oxide, or a crystal plane equivalent thereto. The metal oxide 9 is preferably one in which the area ratio of the (111) plane is the largest among all crystal planes detected in the metal oxide 9. The atomic density on the crystal plane of the magnesium oxide in contact with the PEEK resin 8 is preferably 17 atoms/nm² or more.

According to the metal oxides 9 of (4) to (6), the relative difference between the distance between the benzene rings constituting the molecular chain of the PEEK resin and the distance between the metal atoms constituting the crystal structure of the metal oxide is 5% or less. Therefore, by the action of aligning the atomic arrangement of the PEEK resin, a lattice-matched interface can be formed between the PEEK resin and the metal oxide, and the adhesion strength between them can be increased. Further, in a region away from the interface, the adhesion strength between the molecular chains of the PEEK resin can be increased, and the mechanical properties such as the yield strength of the base material formed of the PEEK resin can be improved.

FIG. 5 is a perspective view schematically showing a metal oxide dispersed in the composite material according to the embodiment of the present invention. FIG. 5 shows a single crystal metal oxide in which the structure of the crystal lattice is reflected on the surface. In FIG. 5, reference numerals 10 to 13 denote crystal planes of the metal oxide, reference numeral s denotes the longitudinal length of the crystal of the metal oxide, reference numeral t denotes the transverse length of the crystal of the metal oxide, and reference numeral h denotes the height of the crystal of the metal oxide.

In FIG. 5, when the crystal plane 10 or the crystal plane 11 is the crystal plane having the largest area among the surfaces of the metal oxide 9 dispersed in the composite material 7, it is preferable that the relative difference between the distance between the benzene rings constituting the molecular chain of the PEEK resin 8 and the distance between the metal atoms constituting the crystal structure of the metal oxide 9 is 5% or less on the crystal plane 10 or the crystal plane 11.

For example, when the metal oxide 9 is a single crystal of titanium dioxide in the rutile phase, the crystal lattice is tetragonal. In such a case, it is preferable that the crystal plane 10 or the crystal plane 11 having the largest area is the (110) plane. Further, it is preferable that the crystal plane 12 or the crystal plane 13 perpendicular to the crystal plane 10 or the crystal plane 11 is the (1-10) plane. This is because the (1-10) plane has the same atomic arrangement as the (110) plane and forms a lattice-matched interface with the PEEK resin 8.

Further, when the metal oxide 9 is a single crystal of α-aluminum oxide which is hexagonal, it is preferable that the crystal plane having the largest area or the crystal plane parallel to the crystal plane is the (001) plane or a crystal plane equivalent thereto. Further, when the metal oxide 9 is a single crystal of magnesium oxide which is cubic, it is preferable that the crystal plane having the largest area or the crystal plane parallel to the crystal plane is the (111) plane or a crystal plane equivalent thereto.

The crystal orientation and the area ratio of a predetermined crystal plane of the metal oxide can be determined by X-ray diffraction (XRD) measurement. An X-ray diffraction spectrum for each metal oxide can be obtained by powder X-ray diffraction measurement using a powder of the metal oxide as a sample. The crystal orientation of the metal oxide and the area ratio of a predetermined crystal plane can be obtained by calculating the ratio of the peak area of the individual crystal plane represented by a predetermined Miller index to the total sum of the peak areas of all the crystal planes detected in the obtained X-ray diffraction spectrum.

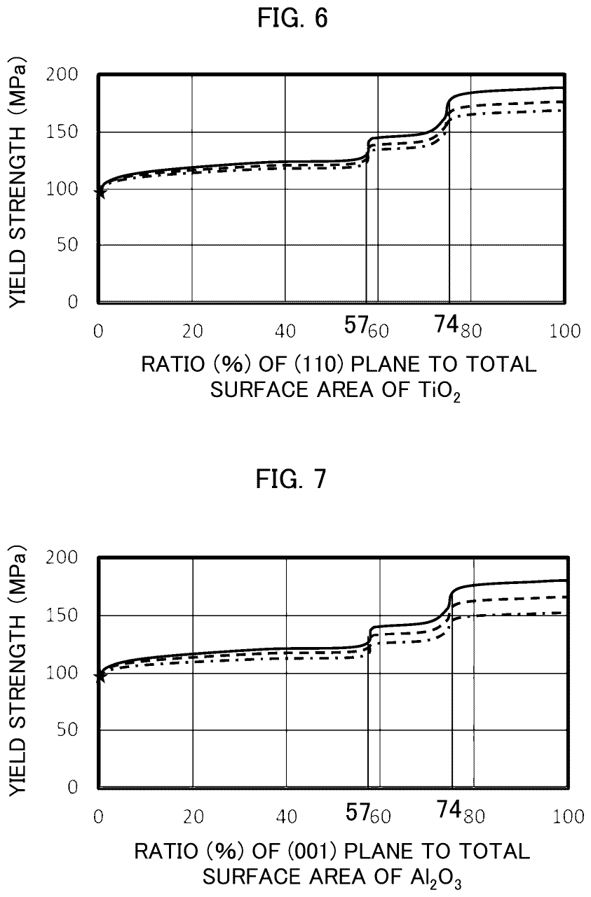

FIG. 6 is a diagram showing analysis results of the relationship between the ratio of the (110) plane to the total surface area of titanium dioxide and the yield strength of the composite material. FIG. 6 shows the relationship between the ratio of the crystal plane having a Miller index of (110) of titanium dioxide in the rutile phase obtained by powder X-ray diffraction measurement and the yield strength of the composite material in which titanium dioxide is blended so as to achieve the ratio. The concentration of titanium dioxide is 0.3 at % in all cases. It was assumed that the (110) plane of titanium dioxide in the rutile phase corresponds to the crystal plane 10 or the crystal plane 11 in FIG. 5.

The solid line in the figure shows the result of changing the ratio of the (110) plane by fixing the longitudinal length s and the transverse length t in FIG. 5 to 8 nm and changing only the height h. The broken line in the figure shows the result of changing the ratio of the (110) plane by fixing the transverse length t and the height h in FIG. 5 to 8 nm and changing only the longitudinal length s. The dashed-dotted line in the figure shows the result of changing the ratio of the (110) plane by fixing the transverse length t to 8 nm and the height h to 4 nm in FIG. 5 and changing only the longitudinal length s.

As shown in FIG. 6, the yield strength of the composite material was relatively high when only the height h was changed. When the ratio of the (110) plane exceeded 57% or 74%, the yield strength increased stepwise. From the viewpoint of improving the yield strength of the composite material, the ratio of the (110) plane in the total surface area of titanium dioxide is preferably 57% or more, and more preferably 74% or more.

FIG. 7 is a diagram showing analysis results of the relationship between the ratio of the (001) plane to the total surface area of aluminum oxide and the yield strength of the composite material. FIG. 7 shows the relationship between the ratio of the crystal plane having a Miller index of (001) of aluminum oxide obtained by powder X-ray diffraction measurement and the yield strength of the composite material in which aluminum oxide is blended so as to achieve the ratio. The concentration of aluminum oxide is 0.3 at % in all cases. It was assumed that the (001) plane of aluminum oxide corresponds to the crystal plane 10 or the crystal plane 11 in FIG. 5.

The solid line in the figure shows the result of changing the ratio of the (001) plane by fixing the longitudinal length s and the transverse length t in FIG. 5 to 8 nm and changing only the height h. The broken line in the figure shows the result of changing the ratio of the (001) plane by fixing the transverse length t and the height h in FIG. 5 to 8 nm and changing only the longitudinal length s. The dashed-dotted line in the figure shows the result of changing the ratio of the (001) plane by fixing the transverse length t to 8 nm and the height h to 4 nm in FIG. 5 and changing only the longitudinal length s.

As shown in FIG. 7, the yield strength of the composite material was relatively high when only the height h was changed. When the ratio of the (001) plane exceeded 57% or 74%, the yield strength increased stepwise. From the viewpoint of improving the yield strength of the composite material, the ratio of the (001) plane to the total surface area of aluminum oxide is preferably 57% or more, and more preferably 74% or more.

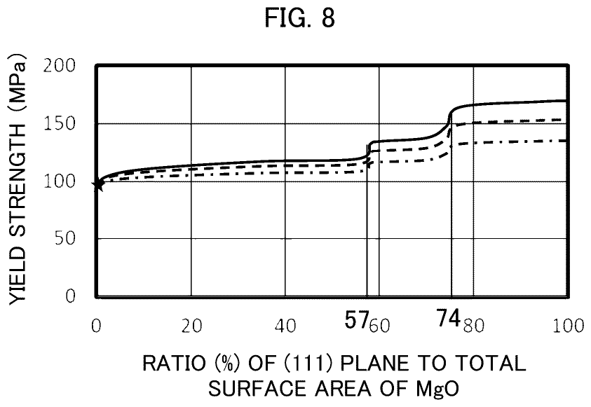

FIG. 8 is a diagram showing analysis results of the relationship between the ratio of the (111) plane to the total surface area of magnesium oxide and the yield strength of the composite material. FIG. 8 shows the relationship between the ratio of the crystal plane having a Miller index of (111) of magnesium oxide obtained by powder X-ray diffraction measurement and the yield strength of the composite material in which magnesium oxide is blended so as to achieve the ratio. The concentration of magnesium oxide is 0.3 at % in all cases. It was assumed that the (111) plane of magnesium oxide corresponds to the crystal plane 10 or the crystal plane 11 in FIG. 5.

The solid line in the figure shows the result of changing the ratio of the (111) plane by fixing the longitudinal length s and the transverse length t in FIG. 5 to 8 nm and changing only the height h. The broken line in the figure shows the result of changing the ratio of the (111) plane by fixing the transverse length t and the height h in FIG. 5 to 8 nm and changing only the longitudinal length s. The dashed-dotted line in the figure shows the result of changing the ratio of the (111) plane by fixing the transverse length t to 8 nm and the height h to 4 nm in FIG. 5 and changing only the longitudinal length s.

As shown in FIG. 8, the yield strength of the composite material was relatively high when only the height h was changed. When the ratio of the (111) plane exceeded 57% or 74%, the yield strength increased stepwise. From the viewpoint of improving the yield strength of the composite material, the ratio of the (111) plane to the total surface area of magnesium oxide is preferably 57% or more, and more preferably 74% or more.

Next, the relationship between the concentration or particle size of the metal oxide and the yield strength of the composite material was analyzed by fixing the area ratio of the crystal plane of the metal oxide having a predetermined distance between metal atoms.

The relationship between the concentration of the metal oxide and the yield strength of the composite material was obtained by fixing the longitudinal length s and the transverse length t in FIG. 5 to 8 nm, changing only the height h, fixing the ratio of the (110) plane of titanium dioxide, the ratio of the (001) plane of aluminum oxide, and the ratio of the (111) plane of magnesium oxide to 74%, and changing the concentration of the metal oxide dispersed in the PEEK resin.

As a result of analyzing the relationship between the concentration of the metal oxide and the yield strength of the composite material, in any of the metal oxides, when the concentration of the metal oxide was 0.2 at % or more and 38 at % or less, the effect of significantly improving the yield strength of the composite material was obtained. It can be said that when the concentration is less than 0.2 at %, the starting points of the action of aligning the atomic arrangement of the PEEK resin are insufficient. Further, if the concentration exceeds 38 at %, the base material formed of the PEEK resin is embrittled and tends to be brittle fractured without exhibiting ductility, which is not preferable.

The relationship between the particle size of the metal oxide and the yield strength of the composite material was obtained by fixing the ratio of the (110) plane of titanium dioxide, the ratio of the (001) plane of aluminum oxide, and the ratio of the (111) plane of magnesium oxide to 74% and changing the average particle size of the metal oxide dispersed in the PEEK resin.

As a result of analyzing the relationship between the particle size of the metal oxide and the yield strength of the composite material, in any of the metal oxides, when the average particle size of the metal oxide was 2 nm or more and 10 μm or less, the effect of significantly improving the yield strength of the composite material was obtained. It can be said that when the average particle size is less than 2 nm, it is difficult to obtain a crystal plane exhibiting the action of aligning the atomic arrangement of the PEEK resin. Further, if the average particle size exceeds 10 μm, the base material formed of the PEEK resin is embrittled and tends to be brittle fractured without exhibiting ductility, which is not preferable.

Next, a method of manufacturing the composite material according to the present embodiment containing a metal oxide with PEEK resin as a base material will be described.

The composite material according to the present embodiment can be manufactured by a manufacturing method of dispersing a predetermined metal oxide exhibiting an action of aligning the atomic arrangement of the PEEK resin in a base material formed of the PEEK resin. The metal oxide is blended with the PEEK resin as a filler having a predetermined crystal structure so that the relative difference between the distance between the benzene rings of the PEEK resin and the distance between the metal atoms of the metal oxide is 5% or less.

The method for manufacturing the composite material according to the present embodiment includes a heat kneading step of heating and kneading a mixture of PEEK resin and a metal oxide, and a molding step of hot-molding the resin composition obtained in the heat kneading step to improve the crystal orientation of the metal oxide.

The PEEK resin can be prepared in an appropriate raw material form such as pellets or powder. The metal oxide can be prepared in a raw material form such as powder. The raw material PEEK resin and the metal oxide can be mixed with additives added as necessary. A mixture of these raw materials is kneaded while being heated and melted to obtain a resin composition.

The resin composition can be kneaded using various apparatuses such as a batch type closed kneading machine such as a Banbury mixer or a pressure kneader, an open type kneading machine such as a roll type kneading machine or a rotor type kneading machine, and a single-screw extruder or a twin-screw extruder. The heating conditions are preferably about 373 °C±20 °C for about 2 minutes. Under such heating conditions, the PEEK resin can be fluidized without being thermally decomposed and uniformly mixed with the metal oxide.

Subsequently, the resin composition is hot-molded to form a molded product in which the crystal orientation of the metal oxide is improved. The molding temperature is preferably 100 °C or more and 400 °C or less. When the molding temperature is 100 °C or more, it is possible to reduce disorder of the crystal structure of the metal oxide and to increase the area ratio of the predetermined crystal structure in which the distance between metal atoms is a predetermined length. When the molding temperature is 400 °C or less, thermal decomposition of the PEEK resin can be avoided.

The resin composition can be molded into an arbitrary shape depending on the application of the composite material. As a molding method, an appropriate method such as extrusion molding, injection molding, blow molding, compression molding, or additive manufacturing can be used. When a mold is used for molding the resin composition, the mold temperature is preferably set to 100 °C or more in terms of reducing disorder of the crystal structure of the metal oxide.

According to the composite material according to the present embodiment described above, the relative difference between the distance between the benzene rings of the PEEK resin and the distance between the metal atoms of the metal oxide is 5% or less, and the metal oxide exhibiting an action of aligning the atomic arrangement of the PEEK resin is dispersed in the base material formed of the PEEK resin, so that a lattice-matched interface can be formed between the PEEK resin and the metal oxide. Therefore, a composite material having high adhesion strength between the PEEK resin and the metal oxide can be obtained. Further, since the metal oxide is dispersed in the base material and the action of aligning the atomic arrangement of the PEEK resin also extends to a region away from the interface, the yield strength of the base material formed of the PEEK resin can be improved.

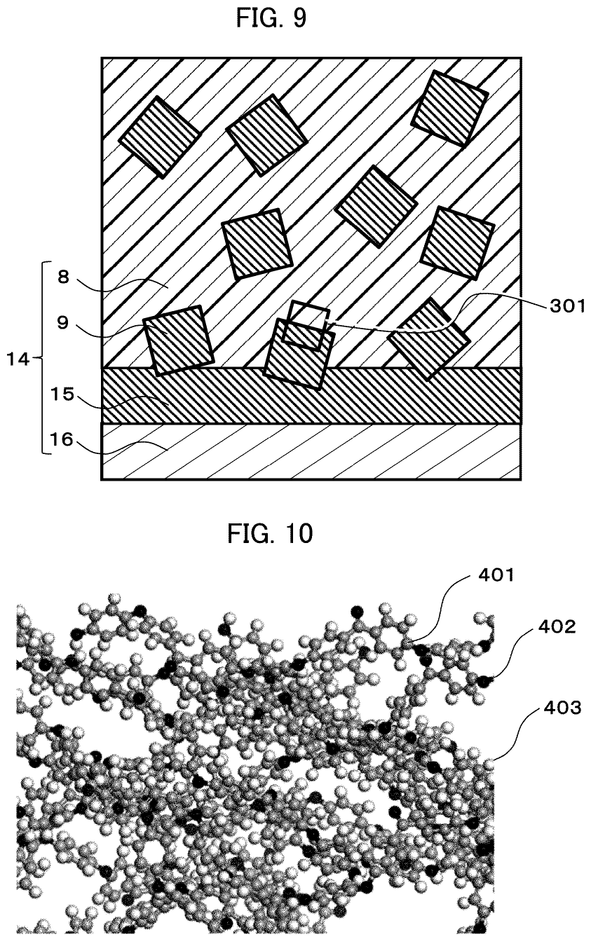

FIG. 9 is a cross-sectional view schematically showing an example of a composite material according to the embodiment of the present invention. FIG. 9 shows an example of a composite material containing a metal oxide as a filler with PEEK resin as a base material and having a metal oxide as a joining material joined to the surface of the PEEK resin. In FIG. 9, reference numeral 8 denotes PEEK resin, reference numeral 9 denotes a metal oxide as a filler, reference numeral 14 denotes a composite material, reference numeral 15 denotes a metal oxide as a joining material, reference numeral 16 denotes a metal, and reference numeral 301 denotes an interface between the PEEK resin and the metal oxide.

As shown in FIG. 9, the composite material 14 according to the present embodiment includes at least PEEK resin 8 forming a base material and a metal oxide 9 dispersed in the base material. As the metal oxide 9, a predetermined metal oxide having a predetermined distance between metal atoms and exhibiting an action of aligning the atomic arrangement of the PEEK resin is blended.

The composite material 14 according to the present embodiment is in the form of a joined material in which a metal oxide 15 as a joining material is joined to the surface of the PEEK resin 8. The metal oxide 15 may constitute the joining material alone, but may constitute the joining material together with the metal 16 as shown in FIG. 9. The metal oxide 15 may be formed by a method of thermally oxidizing a part of the surface side of the metal 16 or by a method of forming a film on the surface of the metal 16.

As the metal oxide 15 as the joining material, an oxide of an appropriate metal can be used as in the case of the metal oxide 3. As the metal oxide 15, a single crystal or a polycrystal may be used. The thickness of the metal oxide 15 in the direction perpendicular to the surface joined to the PEEK resin 8 is preferably 2 nm or more and 100 μm or less. The atomic density on the crystal plane of the metal oxide 15 joined to the PEEK resin 8 is preferably 17 atoms/nm² or more.

As the metal 16 constituting the joining material together with the metal oxide 15, an appropriate pure metal or alloy can be used. The metal 16 can be processed and molded into an arbitrary shape according to the application of the composite material 14. From the viewpoint of reducing the weight of the composite material 14, the metal 16 is preferably a light metal having a specific gravity smaller than that of iron, and is preferably titanium, aluminum, magnesium, or an alloy thereof. The metal oxide 15 and the metal 16 are preferably made of the same kind of metal. Such a metal oxide 15 can be formed by a method of thermally oxidizing the surface of the metal 16.

In the composite material 14 according to the present embodiment, preferred examples of the metal oxide 15 joined to the PEEK resin 8 and the metal oxide 9 dispersed in the base material formed of the PEEK resin 8 are as follows (7) to (9).
(7) The metal oxide 15 as the joining material is titanium dioxide (titania: TiO₂). The crystal plane of the metal oxide 15 joined to the PEEK resin 8 is a crystal plane having a Miller index of (110) of titanium dioxide having a rutile structure, or a crystal plane equivalent thereto. The atomic density on the crystal plane of the titanium dioxide having a rutile structure joined to the PEEK resin 8 is preferably 17 atoms/nm² or more. The metal oxide 9 as the filler is preferably titanium dioxide. The titanium dioxide is preferably formed on the surface of titanium or a titanium alloy.
(8) The metal oxide 15 as the joining material is aluminum oxide (alumina: Al₂O₃). The crystal plane of the metal oxide 15 joined to the PEEK resin 8 is a crystal plane having a Miller index of (001) of aluminum oxide, or a crystal plane equivalent thereto. The atomic density on the crystal plane of the aluminum oxide joined to the PEEK resin 8 is preferably 17 atoms/nm² or more. The metal oxide 9 as the filler is preferably aluminum oxide. The aluminum oxide is preferably formed on the surface of aluminum or an aluminum alloy.
(9) The metal oxide 15 as the joining material is magnesium oxide (magnesia: MgO). The crystal plane of the metal oxide 15 joined to the PEEK resin 8 is a crystal plane having a Miller index of (111) of magnesium oxide, or a crystal plane equivalent thereto. The atomic density on the crystal plane of the magnesium oxide joined to the PEEK resin 8 is preferably 17 atoms/nm² or more. The metal oxide 9 as the filler is preferably magnesium oxide. The magnesium oxide is preferably formed on the surface of magnesium or a magnesium alloy.

According to the combinations of (7) to (9), the yield strength of the composite material 7 is increased by lattice matching at the interface 301 between the PEEK resin 8 and the metal oxide 9, and the joining strength between the PEEK resin 8 or the metal oxide 9 and the metal oxide 14 is increased by lattice matching at the interface between the composite material 7 and the metal oxide 14. Further, by forming the metal oxide 14 by thermal oxidation of the metal 15, the joining strength between the metal oxide 14 and the metal 15 can be increased. Therefore, a material in which the composite material 7 having high yield strength and the metal 15 are combined with high joining strength can be obtained.

According to the composite material according to the present embodiment described above, the relative difference between the distance between the benzene rings of the PEEK resin and the distance between the metal atoms of the metal oxide is 5% or less, and the metal oxide exhibiting an action of aligning the atomic arrangement of the PEEK resin is dispersed in the base material formed of the PEEK resin and joined to the PEEK resin, so that the yield strength of the base material formed of the PEEK resin and the adhesion strength between the PEEK resin and the metal oxide are high, and a composite material that hardly causes interfacial failure at the interface between the PEEK resin and the metal oxide can be obtained.

Further, by joining PEEK resin to a metal or a metal oxide, a structural material or the like can be multi-materialized. Multi-materialization with a lightweight metal or metal oxide makes it possible to reduce the weight of a mobile object or the like. Since the fuel efficiency and power efficiency of the mobile object can be improved, the environmental load can be reduced toward carbon neutrality. Further, the material of the implant can be multi-materialized to reduce the weight and improve the multifunctionality of the implant.

Multi-materialization means manufacturing a product that has been manufactured using any one of a metal material, an inorganic material, a resin material, etc. as a material by combining multiple types of materials. When reducing the weight of a product, a method of replacing a conventionally used metal material or inorganic material with a resin material is effective. On the other hand, from the viewpoints of workability, processing accuracy, moldability, and the like, a metal material or an inorganic material may be advantageous over a resin material. When a part of a metal material or an inorganic material is replaced with a resin material, a lightweight system or device can be manufactured while ensuring workability, processing accuracy, moldability, and the like.

The joined body in which the PEEK resin and the metal oxide are joined and the composite material containing the metal oxide with the PEEK resin as a base material can be used for appropriate applications such as implant applications and applications other than implants. In FIGS. 1 and 2, the joined body 1 forms an intervertebral cage, but the joined body 1 may be used for other implants or applications other than implants.

Examples of the implant application include spine-related orthopedic implants such as intervertebral cages and pedicle screws, orthopedic implants other than spine-related implants such as artificial joints, artificial bone shafts, and internal fixation devices for fractures, ophthalmic implants such as plates, medical implants such as cosmetic surgery implants, and dental implants such as artificial tooth roots.

Examples of the application other than implants include structural materials forming mobile objects, structural materials and parts forming machines and apparatuses, and the like. Examples of the mobile object include automobiles, construction machines, railroad vehicles, motorcycles, bicycles, wheelchairs, elevators, ships, diving equipment, submarines, aircraft, helicopters, drones, rockets, artificial satellites, space probes, space stations, and space elevators. Examples of the machine or apparatus include those installed outdoors and those requiring light weight as well as yield strength, ultraviolet resistance, heat resistance, impact resistance, and the like.

Next, the results of comparing the atomic arrangement of PEEK resin not using a metal oxide with the atomic arrangement of PEEK resin using a metal oxide will be shown in order to confirm the action mechanism of the action of the metal oxide to align the atomic arrangement of the PEEK resin.

The atomic arrangement was analyzed by molecular dynamics simulation combined with density functional theory (DFT), as described in Non-Patent Literature 1 (R. Car, M. Parrinello (1985). Unified Approach for Molecular Dynamics and Density-Functional Theory. PHYSICAL REVIEW LETTERS. VOLUME 55, NUMBER 22, 2471-2474). In molecular dynamics simulation combined with DFT, the structure of the target substance is represented by a functional of electron density, and the coordinates of each constituent atom at each time can be calculated from molecular orbitals and energy obtained from equations of motion.

FIG. 10 is a diagram showing analysis results of the atomic arrangement of PEEK resin not using a metal oxide. FIG. 10 shows an example of the atomic arrangement of PEEK resin when a metal oxide exhibiting an action of aligning the atomic arrangement of the PEEK resin is not dispersed or joined. In FIG. 10, gray spheres indicated by reference numeral 401 represent carbon atoms, black spheres indicated by reference numeral 402 represent oxygen atoms, and white spheres indicated by reference numeral 403 represent hydrogen atoms.

As shown in FIG. 10, when a metal oxide exhibiting an action of aligning the atomic arrangement of the PEEK resin is not dispersed or joined, the orientation and conformation of the molecular chains of the PEEK resin are irregular. Even when a crystal phase is formed by the molecular chains of the PEEK resin, the state is poorly ordered. In such a state, the interaction between the molecular chains of the PEEK resin is weak, and high adhesion strength cannot be obtained.

FIG. 11 is a diagram showing analysis results of the atomic arrangement of PEEK resin using titanium dioxide. FIG. 11 shows the results for PEEK resin joined with titanium dioxide (TiO₂) in the rutile phase as the metal oxide. The upper diagram in FIG. 11 is a view of the interface between the PEEK resin and the metal oxide seen from the side, and the lower diagram is a view of the interface between the PEEK resin and the metal oxide seen from below. The interface between the PEEK resin and the metal oxide is the (110) plane of titanium dioxide in the rutile phase.

In FIG. 11, large gray spheres indicated by reference numeral 401 represent carbon atoms of the PEEK resin, large black spheres indicated by reference numeral 402 represent oxygen atoms of the PEEK resin, large white spheres indicated by reference numeral 403 represent hydrogen atoms of the PEEK resin, small gray spheres indicated by reference numeral 501 represent titanium atoms of titanium dioxide, small black spheres indicated by reference numeral 502 represent oxygen atoms of titanium dioxide, and hexagons indicated by reference numeral 503 represent a ring structure formed on the (110) plane of titanium dioxide in the rutile phase.

As shown in FIG. 11, PEEK resin has a molecular structure in which multiple benzene rings are connected via ether groups or ketone groups. The distance between the centers of the benzene rings connected via the ether group of the PEEK resin is about 0.5719 nm.

On the other hand, titanium dioxide in the rutile phase has an 8-membered ring structure formed of titanium atoms and oxygen atoms when viewed from a direction perpendicular to the (110) plane. The distance between titanium atoms forming the ring structure, which are second nearest neighbors on the same straight line, is about 0.5918 nm.

The relative difference between about 0.5719 nm, which is the distance between the benzene rings of the PEEK resin, and about 0.5918 nm, which is the distance between the second nearest titanium atoms constituting the crystal structure of titanium dioxide in the rutile phase, is about 3.48%. The benzene rings of the PEEK resin and the ring structure 503 formed on the (110) plane of titanium dioxide are aligned and overlapped at equal intervals.

In such a combination, the relative difference between the distance between the benzene rings constituting the molecular chain of the PEEK resin and the distance between the metal atoms constituting the crystal structure of the metal oxide is small. A lattice-matched interface is formed between the PEEK resin and the titanium dioxide by periodically arranging specific atoms. It can be seen that the benzene rings of the PEEK resin and the ring structure of the titanium dioxide are stabilized at positions close to each other and form a strong interaction.

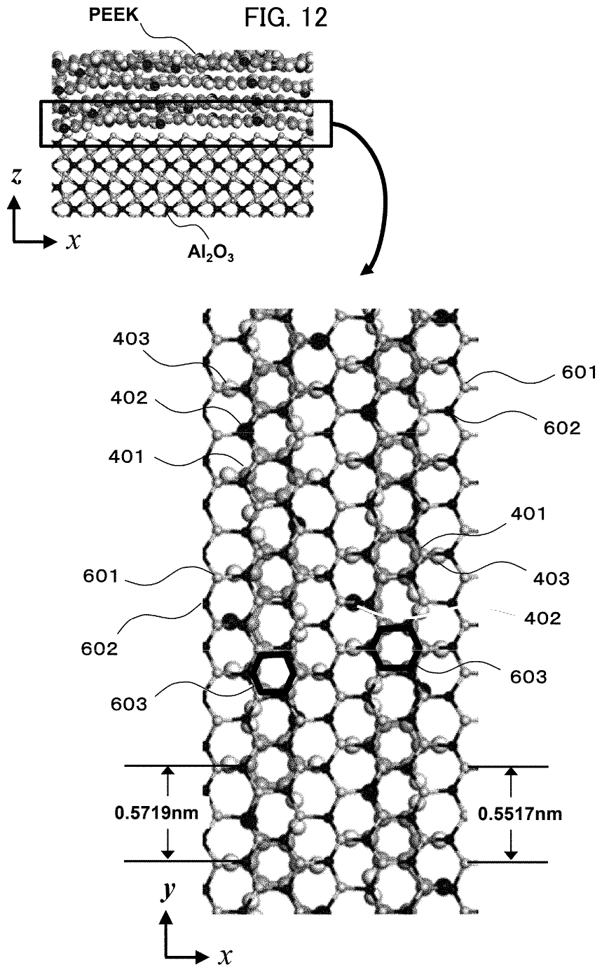

FIG. 12 is a diagram showing analysis results of the atomic arrangement of PEEK resin using aluminum oxide. FIG. 12 shows the results for PEEK resin joined with aluminum oxide (Al₂O₃) as the metal oxide. The upper diagram in FIG. 12 is a view of the interface between the PEEK resin and the metal oxide seen from the side, and the lower diagram is a view of the interface between the PEEK resin and the metal oxide seen from below. The interface between the PEEK resin and the metal oxide is the (001) plane of aluminum oxide.

In FIG. 12, large gray spheres indicated by reference numeral 401 represent carbon atoms of the PEEK resin, large black spheres indicated by reference numeral 402 represent oxygen atoms of the PEEK resin, large white spheres indicated by reference numeral 403 represent hydrogen atoms of the PEEK resin, small gray spheres indicated by reference numeral 601 represent aluminum atoms of aluminum oxide, small black spheres indicated by reference numeral 602 represent oxygen atoms of aluminum oxide, and hexagons indicated by reference numeral 603 represent a ring structure formed on the (001) plane of aluminum oxide.

As shown in FIG. 12, PEEK resin has a molecular structure in which multiple benzene rings are connected via ether groups or ketone groups. The distance between the centers of the benzene rings connected via the ether group of the PEEK resin is about 0.5719 nm.

On the other hand, aluminum oxide has a 6-membered ring structure formed of aluminum atoms and oxygen atoms when viewed from a direction perpendicular to the (001) plane. The distance between aluminum atoms forming the ring structure, which are second nearest neighbors on the same straight line, is about 0.5517 nm.

The relative difference between about 0.5719 nm, which is the distance between the benzene rings of the PEEK resin, and about 0.5517 nm, which is the distance between the second nearest aluminum atoms constituting the crystal structure of aluminum oxide, is about 3.53%. The benzene rings of the PEEK resin and the ring structure 603 formed on the (001) plane of aluminum oxide are aligned and overlapped at equal intervals.

In such a combination, the relative difference between the distance between the benzene rings constituting the molecular chain of the PEEK resin and the distance between the metal atoms constituting the crystal structure of the metal oxide is small. A lattice-matched interface is formed between the PEEK resin and the aluminum oxide by periodically arranging specific atoms. It can be seen that the benzene rings of the PEEK resin and the ring structure of the aluminum oxide are stabilized at positions close to each other.

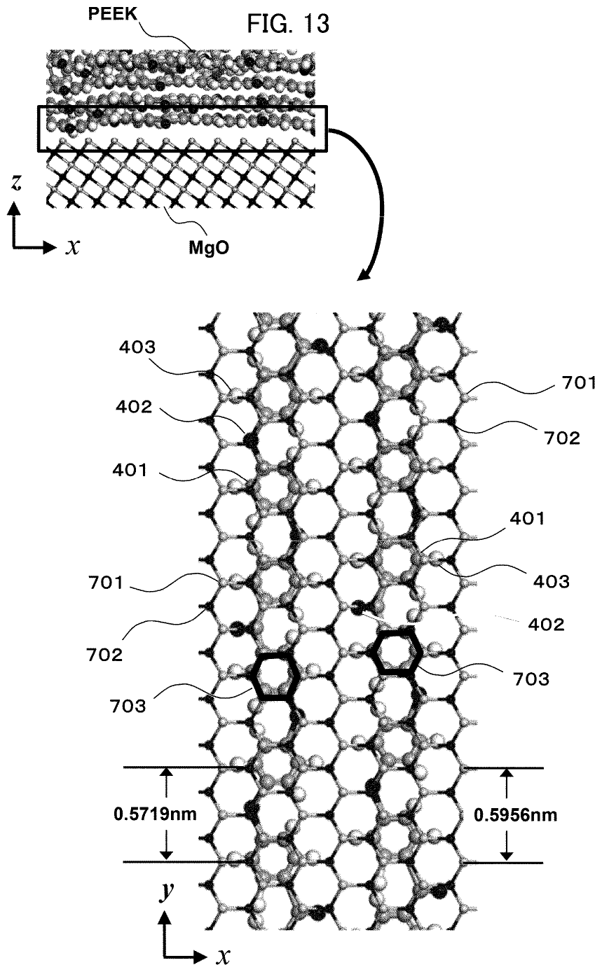

FIG. 13 is a diagram showing analysis results of the atomic arrangement of PEEK resin using magnesium oxide. FIG. 13 shows the results for PEEK resin joined with magnesium oxide (MgO) as the metal oxide. The upper diagram in FIG. 13 is a view of the interface between the PEEK resin and the metal oxide seen from the side, and the lower diagram is a view of the interface between the PEEK resin and the metal oxide seen from below. The interface between the PEEK resin and the metal oxide is the (111) plane of magnesium oxide.

In FIG. 13, large gray spheres indicated by reference numeral 401 represent carbon atoms of the PEEK resin, large black spheres indicated by reference numeral 402 represent oxygen atoms of the PEEK resin, large white spheres indicated by reference numeral 403 represent hydrogen atoms of the PEEK resin, small gray spheres indicated by reference numeral 701 represent magnesium atoms of magnesium oxide, small black spheres indicated by reference numeral 702 represent oxygen atoms of magnesium oxide, and hexagons indicated by reference numeral 703 represent a ring structure formed on the (111) plane of magnesium oxide

As shown in FIG. 13, PEEK resin has a molecular structure in which multiple benzene rings are connected via ether groups or ketone groups. The distance between the centers of the benzene rings connected via the ether group of the PEEK resin is about 0.5719 nm.

On the other hand, magnesium oxide has a 6-membered ring structure formed of magnesium atoms and oxygen atoms when viewed from a direction perpendicular to the (111) plane. The distance between magnesium atoms forming the ring structure, which are second nearest neighbors on the same straight line, is about 0.5956 nm.

The relative difference between about 0.5719 nm, which is the distance between the benzene rings of the PEEK resin, and about 0.5956 nm, which is the distance between the second nearest magnesium atoms constituting the crystal structure of magnesium oxide, is about 4.14%. The benzene rings of the PEEK resin and the ring structure 703 formed on the (111) plane of magnesium oxide are aligned and overlapped at equal intervals.

In such a combination, the relative difference between the distance between the benzene rings constituting the molecular chain of the PEEK resin and the distance between the metal atoms constituting the crystal structure of the metal oxide is small. A lattice-matched interface is formed between the PEEK resin and the magnesium oxide by periodically arranging specific atoms. It can be seen that the benzene rings of the PEEK resin and the ring structure of the magnesium oxide are stabilized at positions close to each other.

As shown in the upper diagrams of FIGS. 11, 12, and 13, when the PEEK resin and the metal oxide are joined, the base material formed of the PEEK resin forms a regular atomic arrangement as compared with the case where the metal oxide is not used. It can be seen that the metal oxide exhibiting the action of aligning the atomic arrangement of the PEEK resin acts not only on the interface between the PEEK resin and the metal oxide, but also on the molecular chains of the PEEK resin in a region away from the interface.

Tables 4 to 6 show the results of calculating the atomic density [atoms/nm²] and peel energy [J/m²] of the metal oxide at the interface between the PEEK resin and the metal oxide. The atomic density and peel energy of the metal oxide were derived by obtaining a function of the peel energy by the response surface method using data calculated by molecular dynamics simulation combined with density functional theory (DFT).

TABLE 4

| | Titanium Dioxide (TiO₂) | | | |
|---|---|---|---|---|
| | (100) | (001) | (110) | (111) |
| Relative Difference | 3.48 | 13.6 | 3.48 | 4.46 |
| between Distance | | | | |
| between Benzene Rings | | | | |
| of PEEK Resin and | | | | |
| Distance between Metal | | | | |
| Atoms of Metal Oxide | | | | |
| (Lattice Mismatch) [%] | | | | |
| Atomic Density of Metal | 7.36 | 15.5 | 20.8 | 12.3 |
| Oxide [atoms/nm²] | | | | |
| Peel Energy [J/m²] | 0.217 | 0.264 | 0.691 | 0.248 |

As shown in Table 4, the peel energy at the interface between the PEEK resin and titanium dioxide in the rutile phase was more than twice as large when the interface was the (110) plane as compared with the (100) plane, (001) plane, and (111) plane. The same tendency as the actual measurement results by the peel test shown in Table 1 was also confirmed in the molecular orbital calculation using DFT.

When hafnium is added to titanium dioxide in the rutile phase so as to be 8 at %, the relative difference between the distance between the benzene rings of the PEEK resin and the distance between the titanium atoms of titanium dioxide is 5.03%. In this case, the peel energy at the interface between the PEEK resin and the titanium dioxide is reduced by about 39%. Therefore, the relative difference between the distance between the benzene rings of the PEEK resin and the distance between the titanium atoms of the titanium dioxide is preferably 5% or less.

TABLE 5

Aluminum Oxide (Al₂O₃)

| | (100) | (001) | (110) | (210) |
|---|---|---|---|---|
| Relative Difference | 0.350 | 3.53 | 0.350 | 16.8 |
| between Distance | | | | |
| between Benzene Rings | | | | |
| of PEEK Resin and | | | | |
| Distance between Metal | | | | |
| Atoms of Metal Oxide | | | | |
| (Lattice Mismatch) [%] | | | | |
| Atomic Density of Metal | 8.06 | 17.8 | 8.06 | 19.4 |
| Oxide [atoms/nm²] | | | | |
| Peel Energy [J/m²] | 0.237 | 0.643 | 0.237 | 0.206 |

As shown in Table 5, the peel energy at the interface between the PEEK resin and the aluminum oxide was more than twice as large when the interface was the (001) plane as compared with the (100) plane, (110) plane, and (210) plane. The same tendency as the actual measurement results by the peel test shown in Table 2 was also confirmed in the molecular orbital calculation using DFT.

When gallium is added to aluminum oxide so as to be 12 at %, the relative difference between the distance between the benzene rings of the PEEK resin and the distance between the aluminum atoms of aluminum oxide is 5.02%. In this case, the peel energy at the interface between the PEEK resin and the aluminum oxide is reduced by about 37%. Therefore, the relative difference between the distance between the benzene rings of the PEEK resin and the distance between the aluminum atoms of aluminum oxide is preferably 5% or less.

TABLE 6

| | Magnesium Oxide (MgO) | | | |
|---|---|---|---|---|
| | (100) | (001) | (110) | (111) |
| Relative Difference | 6.37 | 6.37 | 4.14 | 4.14 |
| between Distance | | | | |
| between Benzene Rings | | | | |
| of PEEK Resin and | | | | |
| Distance between Metal | | | | |
| Atoms of Metal Oxide | | | | |
| (Lattice Mismatch) [%] | | | | |
| Atomic Density of Metal | 22.5 | 22.5 | 15.9 | 26.0 |
| Oxide [atoms/nm²] | | | | |
| Peel Energy [J/m²] | 0.358 | 0.358 | 0.374 | 0.612 |

As shown in Table 6, the peel energy at the interface between the PEEK resin and the magnesium oxide was 1.5 times or more larger when the interface was the (111) plane than when the interface was the (100) plane, (001) plane, or (110) plane. The same tendency as the actual measurement results by the peel test shown in Table 3 was also confirmed in the molecular orbital calculation using DFT.

When calcium is added to magnesium oxide so as to be 9 at %, the relative difference between the distance between the benzene rings of the PEEK resin and the distance between the magnesium atoms of magnesium oxide is 5.04%. In this case, the peel energy at the interface between the PEEK resin and the magnesium oxide is reduced by about 32%. Therefore, the relative difference between the distance between the benzene rings of the PEEK resin and the distance between the magnesium atoms of magnesium oxide is preferably 5% or less.

However, even when the relative difference between the distance between the benzene rings of the PEEK resin and the distance between the metal atoms of the metal oxide is 5% or less, if the atomic density of the metal oxide close to the PEEK resin is small, the total interaction does not become strong, so that the adhesion strength between the PEEK resin and the metal oxide becomes low.

For example, in the case of the (111) plane of titanium dioxide in the rutile phase, the relative difference between the distance between the benzene rings of the PEEK resin and the distance between the second nearest titanium atoms of titanium dioxide in the rutile phase is about 4.46%. However, the atomic density of the metal oxide on the (111) plane is as small as 12.3 atoms/nm². In such a case, the peel energy at the interface between the PEEK resin and the metal oxide is also as small as 0.248 J/m².

Therefore, in order to increase the adhesion strength between the PEEK resin and the metal oxide, it is important to make the relative difference between the distance between the benzene rings constituting the molecular chain of the PEEK resin and the distance between the metal atoms constituting the crystal structure of the metal oxide 5% or less and to increase the atomic density of the metal oxide at the interface between the PEEK resin and the metal oxide.

The metal oxide may contain an additive element or an impurity element that is inevitably mixed. If the additive element or the impurity element is about 1 wt % or less, lattice mismatch is reduced, so that the adhesion strength between the PEEK resin and the metal oxide can be improved. By doping a heterogeneous metal atom that substitutes part of the metal atoms as the main component, it may be possible to adjust the distance between the metal atoms.

When titanium dioxide is used as the metal oxide, it is preferable to include an element having an atomic radius smaller than that of titanium as the additive element or the impurity element. Examples of such an element include aluminum and vanadium.

The average interatomic distance between titanium atoms in pure titanium dioxide is larger than the distance between benzene rings of PEEK resin. When an element having an atomic radius smaller than that of titanium is included, the average interatomic distance of titanium dioxide can be reduced, and the lattice mismatch can be further reduced. For example, when a composite oxide containing 90 wt % of Ti, 6 wt % of Al, and 4 wt % of V is used, the lattice mismatch becomes zero, and the adhesion strength between the PEEK resin and the metal oxide becomes high.

When aluminum oxide is used as the metal oxide, it is preferable to include an element having an atomic radius larger than that of aluminum as the additive element or the impurity element. Examples of such an element include magnesium and titanium.

The average interatomic distance between aluminum atoms in pure aluminum oxide is smaller than the distance between benzene rings of PEEK resin. When an element having an atomic radius larger than that of aluminum is included, the average interatomic distance of aluminum oxide can be increased, and the lattice mismatch can be further reduced. For example, when a composite oxide containing 98.5 wt % of Al and 1.5 wt % of Mg is used, the lattice mismatch becomes zero, and the adhesion strength between the PEEK resin and the metal oxide becomes high.

When magnesium oxide is used as the metal oxide, it is preferable to include an element having an atomic radius smaller than that of magnesium as the additive element or the impurity element. An example of such an element is aluminum.

The average interatomic distance between magnesium atoms in pure magnesium oxide is larger than the distance between benzene rings of PEEK resin. When an element having an atomic radius smaller than that of magnesium is included, the average interatomic distance of magnesium oxide can be reduced, and the lattice mismatch can be further reduced. For example, when a composite oxide containing 90 wt % of Mg and 10 wt % of Al is used, the lattice mismatch becomes zero, and the adhesion strength between the PEEK resin and the metal oxide becomes high.

Although the embodiments of the present invention have been described above, the present invention is not limited to the above-described embodiments, and various modifications are included without departing from the technical scope. For example, the embodiments are not necessarily limited to those having all the configurations described. Further, it is possible to replace a part of the configuration of a certain embodiment with another configuration, or to add another configuration to the configuration of a certain embodiment. It is also possible to add, delete, or replace a part of the configuration of a certain embodiment with another configuration.

For example, in the joined body 1, the metal oxide 3 is joined, but in the joined body in which the PEEK resin and the metal oxide are joined to each other, the metal oxide may be joined to another metal together with the PEEK resin. Such a metal oxide can be formed by a method of thermally oxidizing a part of the surface side of the metal or a method of forming a film on the surface of the metal, as in the case of the composite material 14.

### REFERENCE SIGNS LIST

1 Joined body
2, 8 PEEK resin
3, 9, 15 Metal oxide
4 Vertebral body
5 Intervertebral disc
6 Bone marrow
7, 14 Composite material
10, 11, 12, 13 Crystal plane
16 Metal
101, 201, 301 Interface
102 HAp film
401 Carbon atom
402 Oxygen atom
403 Hydrogen atom
501 Titanium atom
601 Aluminum atom
701 Magnesium atom
502, 602, 702 Oxygen atom
503, 603, 703 Ring structure

1. A joined body in which a polyether ether ketone resin and a metal oxide are joined, wherein
a relative difference between a distance between benzene rings constituting a molecular chain of the polyether ether ketone resin and a distance between metal atoms constituting a crystal structure of the metal oxide is 5% or less; and
an atomic density of the metal oxide on a crystal plane joined to the polyether ether ketone resin is 17 atoms/nm² or more.

2. The joined body according to claim 1, wherein
the metal oxide is TiO₂; and
the crystal plane joined to the polyether ether ketone resin is a (110) plane of TiO₂ having a rutile structure.

3. The joined body according to claim 1, wherein
the metal oxide is Al₂O₃; and
the crystal plane joined to the polyether ether ketone resin is a (001) plane of Al₂O₃.

4. The joined body according to claim 1, wherein
the metal oxide is MgO; and
the crystal plane joined to the polyether ether ketone resin is a (111) plane of MgO.

5. A composite material comprising a metal oxide with a polyether ether ketone resin as a base material, wherein
a relative difference between a distance between benzene rings constituting a molecular chain of the polyether ether ketone resin and a distance between metal atoms constituting a crystal structure of the metal oxide is 5% or less.

6. The composite material according to claim 5, wherein
the metal oxide is TiO₂; and
57% or more of a surface of the metal oxide in contact with the polyether ether ketone resin is a (110) plane of TiO₂ having a rutile structure.

7. The composite material according to claim 5, wherein
TiO₂ is joined to a surface of the polyether ether ketone resin.

8. The composite material according to claim 5, wherein
the metal oxide is Al₂O₃; and
57% or more of a surface of the metal oxide in contact with the polyether ether ketone resin is a (001) plane of Al₂O₃.

9. The composite material according to claim 5, wherein
Al₂O₃ is joined to a surface of the polyether ether ketone resin.

10. The composite material according to claim 5, wherein
the metal oxide is MgO; and
57% or more of a surface of the metal oxide in contact with the polyether ether ketone resin is a (111) plane of MgO.

11. The composite material according to claim 5, wherein
MgO is joined to a surface of the polyether ether ketone resin.

12. An implant formed of the joined body according to claim 1.

13. A structural material for medical use, aerospace use, or vehicle use, formed of the composite material according to claim 5.

14. A method for manufacturing a joined body in which a polyether ether ketone resin and a metal oxide are joined, the method comprising:
a step of film-forming a metal oxide on a surface of a polyether ether ketone resin; and
a step of improving crystal orientation of the metal oxide by heat-treating the polyether ether ketone resin on which the metal oxide is film-formed, wherein
a relative difference between a distance between benzene rings constituting a molecular chain of the polyether ether ketone resin obtained in the step of improving the crystal orientation and a distance between metal atoms constituting a crystal structure of the metal oxide is 5% or less; and
an atomic density of the metal oxide on a crystal plane joined to the polyether ether ketone resin is 17 atoms/nm² or more.

15. A method for manufacturing a composite material containing a metal oxide with a polyether ether ketone resin as a base material, the method comprising:
a step of heating and kneading a mixture of a polyether ether ketone resin and a metal oxide; and
a step of hot-molding a resin composition obtained in the heating and kneading step to improve crystal orientation of the metal oxide, wherein
a relative difference between a distance between benzene rings constituting a molecular chain of the polyether ether ketone resin obtained in the step of improving the crystal orientation and a distance between metal atoms constituting a crystal structure of the metal oxide is 5% or less.
